(19) United States
(12) Patent Application Publication
(10) Pub. No.: US 2017/0059707 A1
(43) Pub. Date: Mar. 2, 2017

(54) Miniature acoustic leaky-wave antenna for ultrasonic imaging

(71) Applicant: The Government of the United States of America, as represented by the Secretary of the Navy, Arlington, VA (US)

(72) Inventors: Charles Alan Rohde, Alexandria, VA (US); Matthew David Guild, Alexandria, VA (US); Christina Jeanne Naify, Pasadena, CA (US)

(21) Appl. No.: 15/246,798

(22) Filed: Aug. 25, 2016

Related U.S. Application Data
(60) Provisional application No. 62/212,654, filed on Sep. 1, 2015.

(51) Int. Cl. G01S 15/89; A61B 8/08; A61B 8/00; A61B 8/12

(52) U.S. Cl. CPC G01S 15/899; A61B 8/12; A61B 8/0891; A61B 8/445; A61B 8/4494; A61B 8/0841; A61B 2017/3413

(57) ABSTRACT

An ultrasonic imaging system includes a micro-acoustic source configured to generate a broadband ultrasonic pulse. The ultrasonic imaging system further includes an acoustic leaky-wave antenna configured to use a frequency dependent angular dispersion to simultaneously collect reflected signals from multiple angles of the broadband ultrasonic pulse, wherein the reflected signals contain information about a surrounding medium. The ultrasonic imaging system further includes a sensor operationally coupled to the acoustic leaky-wave antenna, the sensor configured to detect the reflected signals collected by the acoustic leaky-wave antenna.

### CROSS-REFERENCE TO RELATED APPLICATIONS

This application claims priority to U.S. Provisional Patent Application No. 62/212,654 filed on Sep. 1, 2015, the complete disclosure of which, in its entirety, is herein incorporated by reference.

### GOVERNMENT INTEREST

The embodiments described herein may be manufactured, used, and/or licensed by or for the United States Government without the payment of royalties thereon.

### BACKGROUND

Technical Field

The embodiments herein generally relate to diagnostic detecting tools, and more particularly to ultrasonic imaging tools.

Description of the Related Art

Ultrasonic imaging is a heavily leveraged diagnostic tool in the medical industry, particularly in the fields of cardiology, obstetrics, surgery, and neurology. However, the current state of the art has several complications that limit the use of ultrasonic imaging as a point-of-care diagnostic tool. The complexity and power requirements and external (e.g., outside the body) nature of current systems limit their portability and penetration depth. Typical penetration depths for external ultrasonic imaging techniques limit their useful frequency range, and hence, their resolution. It is desirable to increase the range and versatility of the ultrasonic imaging devices.

### SUMMARY

In view of the foregoing, an embodiment herein provides an ultrasonic imaging system comprising a micro-acoustic source configured to generate a broadband ultrasonic pulse; an acoustic leaky-wave antenna configured to use a frequency dependent angular dispersion to simultaneously collect reflected signals from multiple angles of the broadband ultrasonic pulse, wherein the reflected signals contain information about a surrounding medium; and a sensor operationally coupled to the acoustic leaky-wave antenna, the sensor configured to detect the reflected signals collected by the acoustic leaky-wave antenna.

The ultrasonic pulse generated by the micro-acoustic source may be in the 1-20 MHz ultrasound range. The micro-acoustic source may be communicatively coupled to the leaky-wave antenna. The acoustic leaky-wave antenna, the micro-acoustic source, and the sensor may be configured to be placed inside a vein, and wherein the reflected signals collected by the leaky-wave antenna may be reflected from any of a sidewall of the vein and an object outside the vein. The sensor may comprise a fiber Bragg grating configured to sense pressure fields. The fiber Bragg grating may be configured to generate an optical signal in response to detecting the reflected signals collected by the leaky-wave antenna.

The ultrasonic imaging system may further comprise an optical signal converter optically coupled to the sensor, wherein the optical signal converter may be configured to convert the optical signal generated by the sensor to an electric signal. The ultrasonic imaging system may further comprise a computing device electronically coupled to the optical converter, wherein the computing device may be configured to process and display the information about the surrounding medium in the reflected signals. The sensor may comprise a capacitive micromachined ultrasonic transducer configured to generate an electric signal in response to detecting the reflected signals collected by the acoustic leaky-wave antenna.

Another embodiment herein provides a method comprising a micro-acoustic source configured to generate a broadband ultrasonic pulse; an acoustic leaky-wave antenna comprising a waveguide; and a plurality of periodically structured sub-wavelength acoustic ports on the waveguide configured to coherently interact with the broadband ultrasonic pulse, resulting in frequency dependent leakage of the energy of the broadband ultrasonic pulse through a plurality of leaking wavelettes with a fixed, programmed phase relationship into a surrounding medium, wherein the acoustic leaky-wave antenna is configured to use a frequency dependent angular dispersion to simultaneously collect reflected signals from multiple angles of the broadband ultrasonic pulse, wherein the reflected signals contain information about the surrounding medium; and a sensor operationally coupled to the acoustic leaky wave antenna, the sensor configured to detect the reflected signals collected by the acoustic leaky-wave antenna.

The waveguide may comprise a bio-compatible soft polymer stent. The plurality of acoustic ports may be created using femtosecond laser machining to make any of periodic patterned grooves and open cuts. The plurality of acoustic ports may be created using femtosecond laser machining to make periodic holes. The waveguide may comprise a hypodermic needle. The hypodermic needle may comprise a 28 gauge metal needle. The plurality of acoustic ports may comprise holes each having an approximately 100 μm diameter.

Another embodiment herein provides a probe comprising a micro-acoustic source configured to generate a broadband ultrasonic pulse; an acoustic leaky-wave antenna comprising: a waveguide; a plurality of periodically structured sub-wavelength acoustic ports having a shape of any of patterned grooves and holes; and a sensor operationally coupled to the acoustic leaky wave antenna, the sensor configured to detect reflected signals collected by the acoustic leaky-wave antenna.

The sensor may comprise a fiber Bragg grating configured to sense pressure fields. The fiber Bragg grating may be configured to generate an optical signal in response to detecting the reflected signals. The sensor may comprise a capacitive micromachined ultrasonic transducer configured to generate an electric signal in response to detecting the reflected signals collected by the acoustic leaky-wave antenna.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 is a schematic diagram illustration a Leaky Wave Antenna (LWA) according to an embodiment herein;

FIG. 2A is a schematic diagram illustrating a micro-leaky-wave antenna (μLWA) according to an embodiment herein;

FIG. 2B is a schematic diagram illustrating manufacturing periodic structure of a leaky-wave antenna according to an embodiment herein;

FIG. 2C is a schematic diagram illustrating a first type of periodic sidewall pattern of the antenna according to an embodiment herein;

FIG. 2D is a schematic diagram illustrating a second type of periodic sidewall pattern of the antenna according to an embodiment herein;

FIG. 2E is a schematic diagram illustrating a third type of periodic sidewall pattern of the antenna according to an embodiment herein;

FIG. 2F is a schematic diagram illustrating a fourth type of periodic sidewall pattern of the antenna according to an embodiment herein;

FIG. 2G is a schematic diagram illustrating a fifth type of periodic sidewall pattern of the antenna according to an embodiment herein;

FIG. 2H is a schematic diagram illustrating a sixth type of periodic sidewall pattern of the antenna according to an embodiment herein; and

FIG. 3 illustrates an acoustic leaky-wave antenna system according to an embodiment herein.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Ultrasonic images may be formed in one of several modalities or an overlaid combination of discrete modalities. Examples of these imaging types are pulse-echo, Doppler flow imaging elastography, and 4D imaging. Pulse-echo imaging returns structural information, and Doppler imaging returns fluid flow velocities. Elastography is an extension of Doppler imaging where a strong incident pulse induces motion at a tissue interface, and the elastic properties of the moving tissue can be evaluated. Similarly, 4D imaging is an extension of the pulse-echo technique to retrieve time-resolved 3D images of tissue structures.

In the pulse-echo mode, an ultrasonic pulse (typically in the 1-10 MHz frequency range) is sent from an external piezoelectric source into a tissue zone to be imaged. Scattered return pulses (echoes) can be detected due to differences in acoustic impedance between tissue types. These pulse reflections are detected by the same (or similar) external piezoelectric transducer, and the time delay between pulse and echo determines the distance to the various tissue type interfaces. In Doppler flow imaging, changes in reflected frequency content map onto the flow-velocity of particulate laden fluids such as blood.

Imaging may occur when the pulse source has directivity (a “beam”) and that directivity is scanned in space. The beam forming and scanning may be performed mechanically, with a single specially shaped source, or may be performed with a coherently phased array of multiple piezoelectric transducers. Phased arrays may allow for a nearly arbitrary beam shape and direction. Phased arrays, however, may be limited by the number and size of the array elements. The speed of phased array sensing also allows for fast imaging, and the use of multiple arrays allows real-time imaging of 3D structures (i.e., 4D imaging).

Multi-element linear or phased arrays of transducer elements in an ultrasonic imaging device may lead to very high processing bandwidth (typically greater than 100 GHz) requirements, but may require high power and complex processing capabilities. This may decrease the portability and ease-of-use of the imaging device. Also, arrays of elements may generally be limited in size and external coupling complexity when internal imaging is the goal, such as in intravenous ultrasonic imaging.

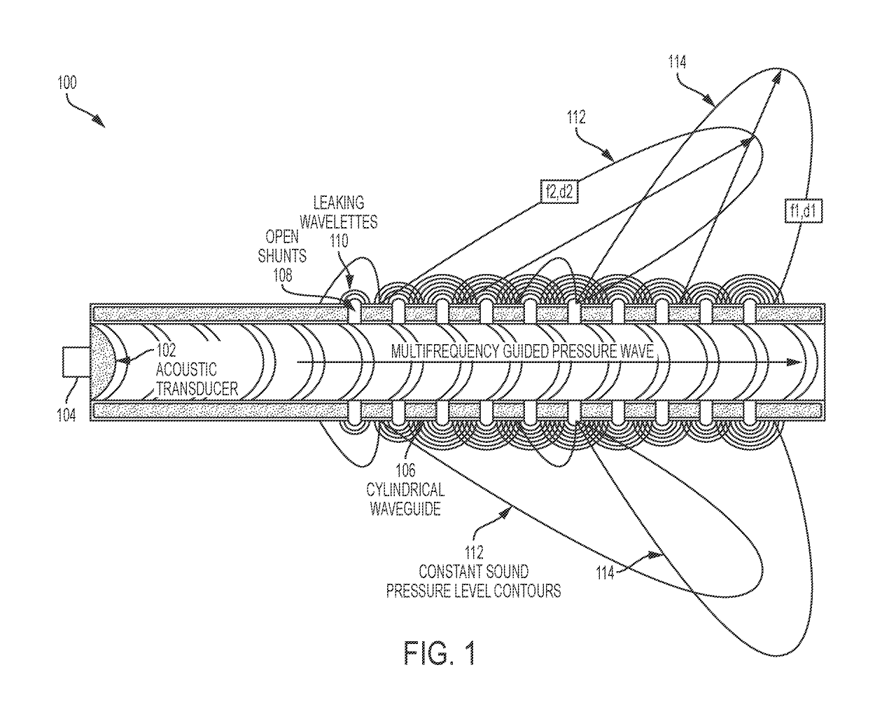

An embodiment herein provides a device that utilizes a geometrically scaled version of an acoustic imaging leaky wave antenna in the 1-20 MHz ultrasound range. In an embodiment, the acoustic imaging leaky wave antenna may be integrated with an output transduction mechanism. In an exemplary embodiment, the output transduction mechanism may be a fiber-optic transducer based on distributed Bragg gratings. To produce a waveguide structure for the high frequency ultrasound aqueous environments, femtosecond laser machining or other micro-machining techniques may be used to engineer optimized, fast-wave-coupling acoustic ports into the surface of a millimeter scale metallic or glass capillary tubes. The probe provided by the embodiments herein may produce 3D ultrasonic images of biological tissue using a single, non-directional source and a single non-directional detector, all in a package sufficiently small to be inserted intravenously, resulting in a robust, arthroscopically enabled tool for interior assessment, surgical monitoring, or structural monitoring in non-biological confined spaces. Referring now to the drawings, and more particularly to FIGS. 1 through 3, where similar reference characters denote corresponding features consistently throughout the figures, there are shown preferred embodiments.

FIG. 1 is a schematic diagram illustrating an acoustic leaky-wave antenna (ALWA) 100. The ALWA 100 uses a frequency dependent angular dispersion relation to simultaneously collect data from multiple angles with a single transducer 102 via a broadband source pulse 104. The ALWA 100 includes a waveguide 106 with periodically structured sub-wavelength output acoustic ports or shunts 108. Acoustic ports 108 may be configured as any of grooves and open cuts. These acoustic ports 108 coherently interact with the guided wave and result in frequency dependent leakage of the guided energy through leaking wavelettes 110 into the surrounding medium. The coherent addition of leaking radiation results in a propagating wave with a frequency dependent direction, for example the propagating wave 112 and the propagating wave 114.

An embodiment herein applies the ideas of leaky wave antenna technology to acoustic analogs. An embodiment herein reduces the size of acoustic leaky-wave antennas, and increases the operational frequency to the ultrasonic imaging range of 1-20 MHz. Accommodating the MHz scale frequencies of ultrasonic imaging reduces the size of the acoustic antenna to the (sub) millimeter scale.

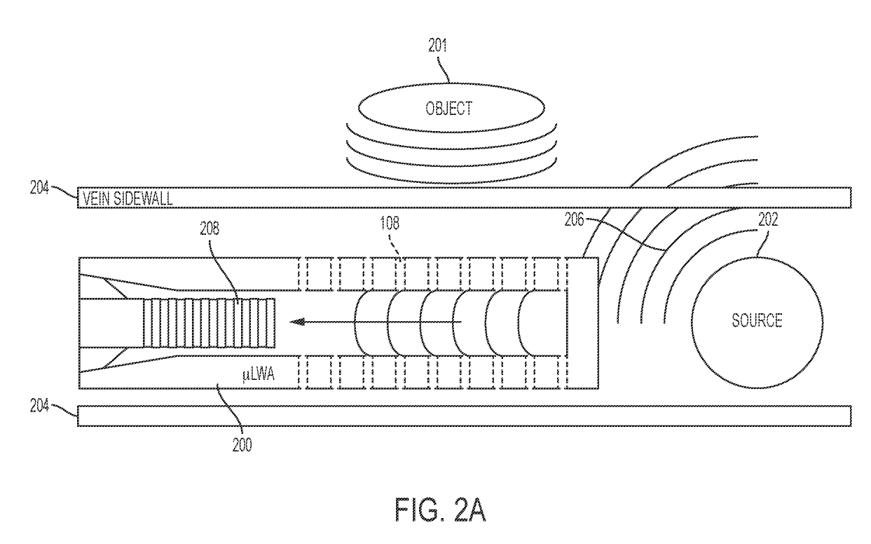

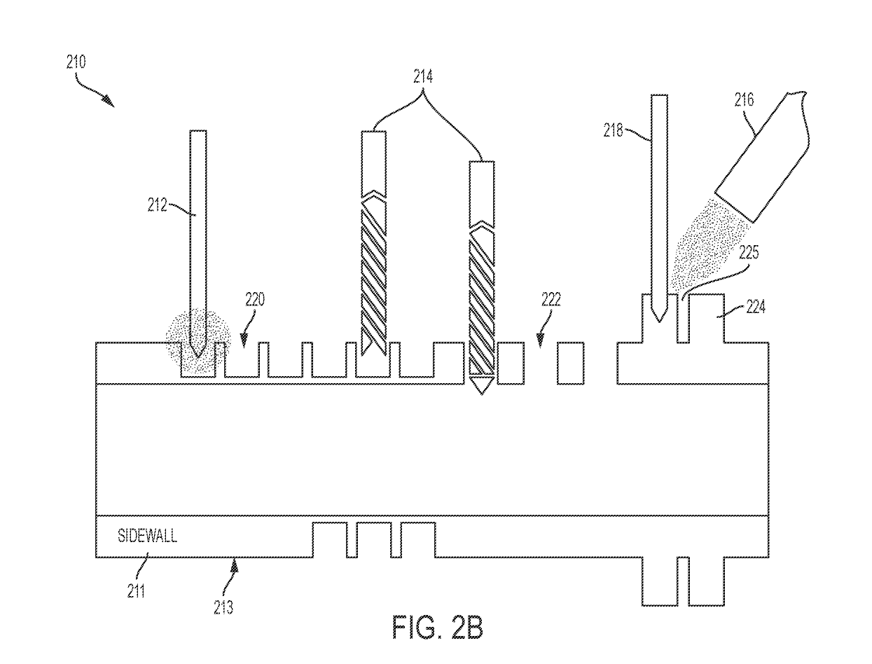

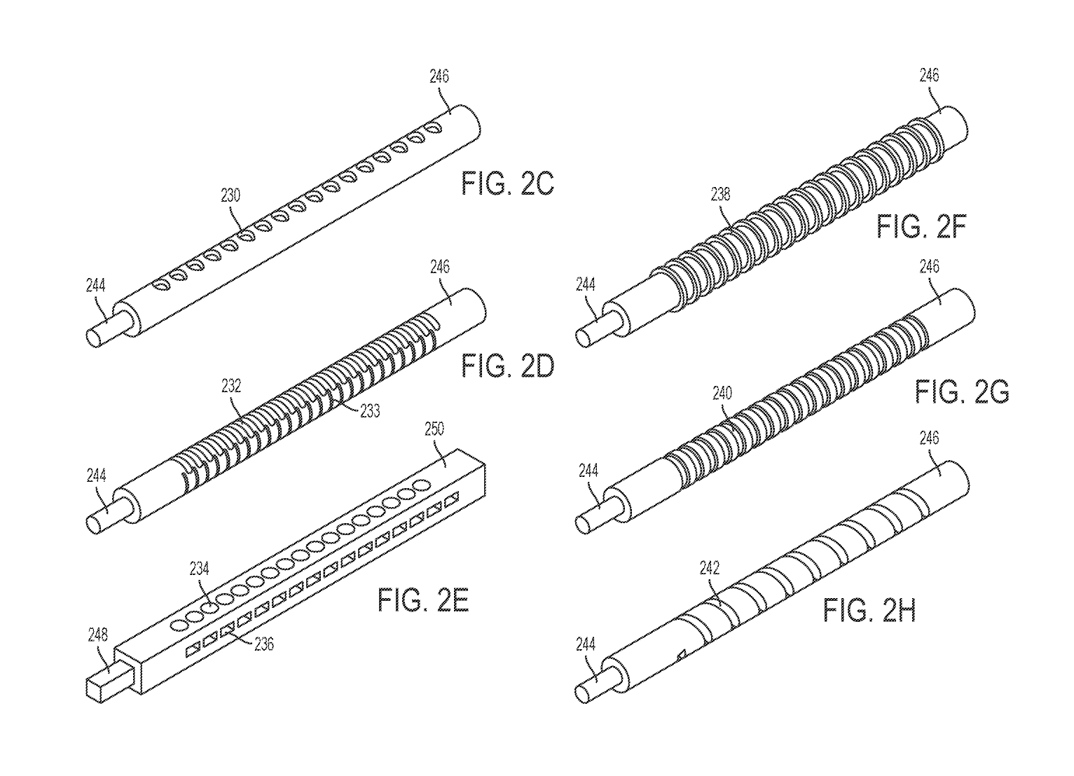

FIG. 2A, with reference to FIG. 1, is a schematic diagram illustrating a micro-leaky-wave antenna (μLWA) 200 according to an embodiment herein. In an embodiment, a micro-acoustic source 202 outputs a high bandwidth ultrasonic pulse 206. In an embodiment, the source 202 may generate ultrasonic pulse in the 1-20 MHz ultrasound range. The micro-acoustic source 202 is communicatively coupled to the μLWA 200. In an embodiment, the micro-acoustic source 202 may be physically attached to the μLWA 200. In an embodiment, the source 202 may be a high powered, broadband source located externally and used to insonifiy a target volume where the uLWA 200 is located. The pulse 206 may illuminate and reflect from the various tissue boundaries, for example vein sidewall 204, or an object 201 located outside the vein and is collected by the micro-acoustic wave antenna 200. In an embodiment, sensor 208 is operationally coupled to the μLWA 200. The sensor 208 may be an acoustic sensor for pressure fields coupled into the μLWA 200. In an embodiment, the sensor 208 is a fiber Bragg grating (FBG). An FBG may be operationally coupled to the μLWA 200, and act as an acoustic sensor for pressure fields coupled into the μLWA 200 through appropriately configured ports.

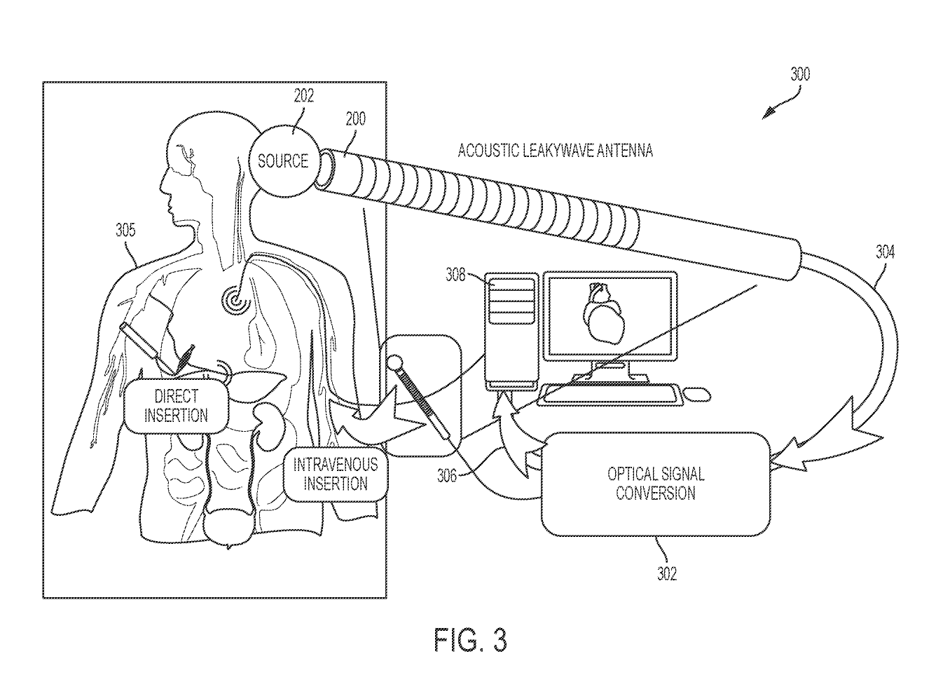

To efficiently convert the collected acoustic signals, fiber optic sensing techniques may be utilized. In an embodiment, by placing the FBG based sensor 208 inside the μLWA 200, interior pressure changes can be monitored and transmitted to an external analysis system (such as computing device 308 shown in FIG. 3). Using an optical signal transport has the advantages of well-developed systems for biological use, very low power consumption, compact design, and high frequency response. The FBG based sensor 208 may be configured based on the homodyne detection of optical phase shifts between a sensing and non-sensing optical path. Detection and 3D imaging of hard or soft object 201 may be conducted by time-resolved (pulse-echo) broadband illumination, short pulse illumination.

In an embodiment, sensor 208 comprises a micro-acoustic sensor such as a capacitive micromachined ultrasonic transducer (CMUT). A CMUT sensor may be used to convert ultrasonic signals into electrical signals. A CMUT could also be used as a micro-acoustic source replacing the source 202.

In an embodiment, CMUTs include a suspended, conductive membrane material above a conductive substrate. Flexure modes of the membrane result in electrical signals through capacitive coupling between the membrane and substrate. CMUTs may be created with a micromechanical machining approach compatible with current 2D photolithography techniques. As such they can be small (micrometer scale), and densely arrayed using current semiconductor processing techniques. Although CMUT elements have no intrinsic directionality, arrays of sensors can be arranged to be directionally sensitive.

FIG. 2B, with reference to FIG. 1 to FIG. 2A, is a schematic diagram illustrating manufacturing periodic structures of a leaky-wave antenna, according to an embodiment herein. In an embodiment, the acoustic ports 108 are manufactured on a sidewall 211 of a longitudinal structure 213. In an embodiment the longitudinal structure 213 may be a cylindrical structure or a rectangular longitudinal structure. In another embodiment the longitudinal structure 213 is a hypodermic needle.

To create the acoustic ports 108 on the μLWA 200, an embodiment utilizes micromachining techniques such as femtosecond laser ablation, using laser beam 212, to create periodic grooves 220 or open holes 222. An embodiment utilizes computer controlled micromachining techniques, using micro mills 214 to create the periodic grooves 220 or the open holes 222. An embodiment uses advanced additive manufacturing techniques, using nozzle 216 and laser beam 218, to make protrusions 224 and periodic groves 225. In an embodiment, periodic grooves 220, 225 or open holes 222 function as the acoustic ports 108.

In an embodiment, femtosecond laser ablation, using laser beam 212, may be used for the high precision removal of material from surface of the structure 213, without heat deposition. This provides the opportunity to machine any material from soft polymers to transparent glasses to metals. Combined with the use of high precision computer numeric control (CNC) multi-axis abrading micro-milling, using micro mills 214 (micro-lathing), femtosecond laser 212, or machining and/or additive welding techniques, using nozzle 216, allow the nearly arbitrary removal/buildup of design material. In an embodiment, the open hole 222 is approximately 100 μm in diameter, bored through metal, 28 gauge hypodermic needle 213. In an embodiment, each of the periodic grooves 220 are approximately 100 μm in diameter.

FIGS. 2C-2H, with reference to FIG. 1 to FIG. 2B, are schematic diagrams illustrating periodic sidewall patterns as the acoustic ports 108 on the μLWA 200, according to exemplary embodiments herein. Any of the manufacturing methods illustrated in FIG. 2B, for example, the laser ablation method, controlled micromachining technique, or the additive manufacturing technique may be used in manufacturing any of the acoustic ports illustrated in FIGS. 2C-2H.

FIG. 2C illustrates periodic holes 230 as the acoustic ports 108. FIG. 2D illustrates multiple slot geometries 232, 233 as the acoustic ports 108. FIG. 2E illustrates periodic slots 234 and divots 236 as the acoustic ports 108. FIG. 2F illustrates periodic extruded ridges 238 as the acoustic ports 108. FIG. 2G illustrates periodic tapered cuts 240 as the acoustic ports 108. FIG. 2H illustrates periodic spiral grooves 242 as the acoustic ports 108.

In the embodiments herein, acoustic ports 230, 232, 234, 236, 238, 240, and 242 are created in a stiff, fluid-filled, hollow material structure 246 having a round cross section, or similar structure 250 having a square cross section. Structures 246, 250 each may have their corresponding transducers 244, 248.

Different patterns shown in exemplary embodiments of FIGS. 2C-2H may have different functionalities. For example, creating slot geometries 232, 233 of FIG. 2D, or periodic slots 234 and divots 236 of FIG. 2E, or continuously varying spiral groove 242 of FIG. 2H, into difference angular regions of the μLWA 200, may produce additional directivity by mapping additional, programmed, frequencies onto the azimuthal quadrants of the μLWA 200.

In some exemplary embodiments, a restricted collection angular field of view is built into the μLWA 200 by only placing acoustic ports in one region of the antenna surface. In an embodiment herein, uniform pattering about the antenna azimuthal surface, such as extruded ridges 238 of FIG. 2F, or periodic tapered cuts 240 of FIG. 2G, may result in a full angular field of view, but no azimuthal signal discrimination. The geometry, size, spacing, depth, sidewall taper of the acoustic ports 108 may depend on the frequency band used by the source 202. In an embodiment the grooves or holes are sub-millimeter in size.

FIG. 3, with reference to FIGS. 1 through 2H, illustrates an acoustic leaky-wave antenna system 300 according to an embodiment herein. In an embodiment, the μLWA 200 and the source 202 may be communicatively connected to an optical signal conversion device 302. The optical signal conversion device 302 is configured to convert an optical signal 304 generated by the μLWA 200 to an electrical signal 306. The electrical signal 306 may be used by a computing device 308 to process and display information detected by the μLWA 200.

In an embodiment, the computing device 308 may map acoustic reflections from soft targets illuminated with an additional non-directional source via their frequency response to an angular position and range. Using external data processing of the optically transduced signal, the computing device 308 may map all objects within the field of view of the μLWA 200. Such a configuration could be operated in any of the currently utilized ultrasonic imaging modalities as discussed above.

In an embodiment, low scale μLWA 200 and source 202 may be inserted intravenously in a body of interest 305 and guided to areas of interest for high-resolution (high-frequency) imaging. The μLWA 200 and source 202 may also be inserted in other tissues or in an organ. Additionally, insertion through small incisions could aid the diagnostic tools available in minimally invasive surgical techniques such as the Da Vinci® robotic surgery available from Intuitive Surgical, Inc., approach or insertion of the device into a traumatic wound entry point could yield a rapid, real time, assessment of interior structure damage near the time of injury. As a non-biological sensor, the μLWA 200 could be used to monitor structural properties of highly confined spaces.

Using the μLWA 200 allows for the use of a single, high bandwidth transducer to acquire signals simultaneously from multiple directions. This single sensor arrangement greatly reduces the complexity and processing needs of an ultrasonic imaging system. In turn, this opens the possibility of creating highly portable diagnostic tools for 3D imaging of organic tissues. Metamaterial techniques also produce LWA designs that make use of negative (left-handed) dispersion relations to extend the field of view of standard LWAs from less than 90° in the forward direction to a full 180°. The use of this additional design parameter space could lead to an even more powerful imaging tool.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. An ultrasonic imaging system comprising:
a micro-acoustic source configured to generate a broadband ultrasonic pulse;
an acoustic leaky-wave antenna configured to use a frequency dependent angular dispersion to simultaneously collect reflected signals from multiple angles of said broadband ultrasonic pulse, wherein said reflected signals contain information about a surrounding medium; and
a sensor operationally coupled to said acoustic leaky-wave antenna, said sensor configured to detect said reflected signals collected by said acoustic leaky-wave antenna.

2. The system of claim 1, wherein said ultrasonic pulse generated by said micro-acoustic source is in a 1-20 MHz ultrasound range.

3. The system of claim 1, wherein said micro-acoustic source is communicatively coupled to said leaky-wave antenna.

4. The system of claim 1, wherein said acoustic leaky-wave antenna, said micro-acoustic source, and said sensor are configured to be placed inside a vein, and wherein said reflected signals collected by said leaky-wave antenna is reflected from any of a sidewall of said vein and an object outside said vein.

5. The system of claim 1, wherein said sensor comprises a fiber Bragg grating configured to sense pressure fields.

6. The system of claim 5, wherein said fiber Bragg grating is configured to generate an optical signal in response to detecting said reflected signals collected by said leaky-wave antenna.

7. The system of claim 1, further comprising an optical signal converter optically coupled to said sensor, wherein said optical signal converter is configured to convert said optical signal generated by said sensor to an electric signal.

8. The system of claim 1, further comprising a computing device electronically coupled to said optical converter, wherein said computing device is configured to process and display said information about said surrounding medium in said reflected signals.

9. The system of claim 1, wherein said sensor comprises a capacitive micromachined ultrasonic transducer configured to generate an electric signal in response to detecting said reflected signals collected by said acoustic leaky-wave antenna.

10. A probe comprising:
a micro-acoustic source configured to generate a broadband ultrasonic pulse;
an acoustic leaky-wave antenna comprising:
a waveguide; and
a plurality of periodically structured sub-wavelength acoustic ports on said waveguide configured to coherently interact with said broadband ultrasonic pulse, resulting in frequency dependent leakage of the energy of said broadband ultrasonic pulse through a plurality of leaking wavelettes with a fixed, programmed phase relationship into a surrounding medium,
wherein said acoustic leaky-wave antenna is configured to use a frequency dependent angular dispersion to simultaneously collect reflected signals from multiple angles of said broadband ultrasonic pulse, wherein said reflected signals contain information about said surrounding medium; and
a sensor operationally coupled to said acoustic leaky wave antenna, said sensor configured to detect said reflected signals collected by said acoustic leaky-wave antenna.

11. The probe of claim 10, wherein said waveguide comprises a bio-compatible soft polymer stent.

12. The probe of claim 10, wherein said plurality of acoustic ports are created using femtosecond laser machining to make any of periodic patterned grooves and open cuts.

13. The probe of claim 10, wherein said plurality of acoustic ports are created using femtosecond laser machining to make periodic holes.

14. The probe of claim 10, wherein said waveguide comprises a hypodermic needle.

15. The probe of claim 14, wherein said hypodermic needle comprises a 28 gauge metal needle.

16. The probe of claim 14, wherein said plurality of acoustic ports comprises holes each having an approximately 100 μm diameter.

17. A probe comprising:
a micro-acoustic source configured to generate a broadband ultrasonic pulse;
an acoustic leaky-wave antenna comprising:
a waveguide;
a plurality of periodically structured sub-wavelength acoustic ports having a shape of any of patterned grooves and holes; and
a sensor operationally coupled to said acoustic leaky wave antenna, said sensor configured to detect reflected signals collected by said acoustic leaky-wave antenna.

18. The probe of claim 17, wherein said sensor comprises a fiber Bragg grating configured to sense pressure fields.

19. The probe of claim 18, wherein said fiber Bragg grating is configured to generate an optical signal in response to detecting said reflected signals.

20. The probe of claim 17, wherein said sensor comprises a capacitive micromachined ultrasonic transducer configured to generate an electric signal in response to detecting said reflected signals collected by said acoustic leaky-wave antenna.
